(19) United States
(12) Patent Application Publication
(10) Pub. No.: US 2013/0109102 A1
(43) Pub. Date: May 2, 2013

(54) LIQUID CHROMATOGRAPHY SYSTEM AND METHOD FOR PROTEIN SEPARATION AND PURIFICATION

(71) Applicants: HIROYUKI YABE, TOKYO (JP); TOMOYUKI MIYABAYASHI, PALATINE, IL (US)

(72) Inventors: MICHAEL LI, GLENVIEW, IL (US); HIROYUKI YABE, TOKYO (JP); TOMOYUKI MIYABAYASHI, PALATINE, IL (US)

(21) Appl. No.: 13/237,326

(22) Filed: Sep. 20, 2011

Related U.S. Application Data
(60) Provisional application No. 61/403,741, filed on Sep. 20, 2010.

(51) Int. Cl. G01N 31/16

(52) U.S. Cl. CPC G01N 31/16
USPC 436/51; 422/75

(57) ABSTRACT

A system and method for protein separation and purification includes a mixing loop, a primary pump having an inlet in communication with a supply of an acid or an alkali and an outlet in communication with the mixing loop. A concentrate pump has an inlet in communication with a supply of an acid or an alkali and an outlet in communication with the mixing loop. A liquid chromatography column is in communication with the mixing loop and is packed with an ion exchange resin and loaded with a protein solution containing a first protein having a first isoelectric point and a second protein having a second isoelectric point. A process control feedback loop in communication with the mixing loop includes an inline process pH analyzer so that a pH of a titration solution in the mixing loop may be detected and adjusted based on the detected pH. The titration solution is provided in the mixing loop by the primary and concentrate pumps and the pH of the titration solution in the mixing loop is adjusted by the feedback loop based on the pH detected by the inline process pH analyzer to provide a generally linear pH gradient to the chromatography column so that the first and second proteins are separately eluded from the liquid chromatography column.

### CLAIM OF PRIORITY

This application claims priority to provisional patent application No. 61/403,741, filed Sep. 20, 2010, currently pending.

### FIELD OF THE INVENTION

The present invention generally relates to liquid chromatography systems and methods and, more particularly, to a system and method where acid and base/alkali liquids are blended in an inline buffer dilution device and in accordance with a method to provide a linear pH gradient during delivery of the blended liquid to a chromatography column loaded with a protein solution for protein separation and purification.

### BACKGROUND

Biopharmaceutical manufacturing requires buffer solutions for critical downstream processes. Chromatography is an integral part of the downstream bioprocessing steps, and the buffered mobile phase properties, such as ionic strength and pH, are often critical process parameters for protein purification. Traditionally, a salt gradient is used to separate proteins with different ionic characteristics. Using this approach, however, protein recovery may be poor and the de-salting steps cumbersome and time-consuming.

Protein separation by pH gradient using prior art systems has proven to be difficult and unpopular. For example, with reference to FIG. 1, a prior art liquid chromatography system consists of two pumps 10 and 12 that independently deliver mobile phase A (acid) and B (base or alkali) to a liquid chromatography column 14. As a result, the percentages of mobile A and mobile B in the blended liquid entering the chromatography column is dependent on the accuracy of the system pumps. The pumps are controlled solely by feedback of the flow rate of the liquids exiting the pumps as detected by flow meters 16 and 18 of FIG. 1.

Use of the prior liquid chromatography system of FIG. 1 generates the sigmoid titration curve of FIG. 2. As illustrated in FIG. 2, during the change of the blended liquid from the acidic to alkaline condition, and vice versa, a steep slope is formed (around 1500 seconds in FIG. 2). Because of the sharp rise in pH during the process, it is very difficult to control the elution characteristics of proteins loaded in the column 14. More specifically, because the titration curve between acid and alkali, whether strong or weak, is not linear, protein chemists are reluctant to use a pH gradient during chromatographic runs to resolve proteins with similar isoelectric points (pI).

In view of the above, and as noted previously, proteins are conventionally purified and separated in a liquid chromatography column by salt gradient (conductivity) and not by pH gradient. Use of conductivity, however, often results in great variances from what is desired. This is often due to human variances in making the buffer solution or variances due to feed stocks from suppliers (for example, 5% off in the salt concentration). Use of a pH gradient in purification/separation of proteins would provide greater control and accuracy, if the above sigmoid titration curve issues could be overcome. It is therefore desirable to make linear pl-I gradients an option for purifying proteins available, for example, in the biopharmaceutical industry.

### BRIEF DESCRIPTION OF THE DRAWINGS

FIG. 1 is a schematic view of a prior art liquid blending and chromatography system;

FIG. 2 is a graph illustrating sigmoid titration curve formed by mixing an acid and an alkali using the prior art system of FIG. 1;

FIG. 3 is schematic view of an embodiment of the liquid chromatography protein separation system of the present invention;

FIG. 4 is a graph illustrating a comparison of titration curves produced by the systems of FIGS. 1 and 3;

FIG. 5 is a graph illustrating elution behavior of two proteins with similar pI under different pH gradient influences from the systems of FIGS. 1 and 3; and

FIG. 6 is a graph illustrating a comparison of curves using a two-solvent embodiment of the system and method of the present invention.

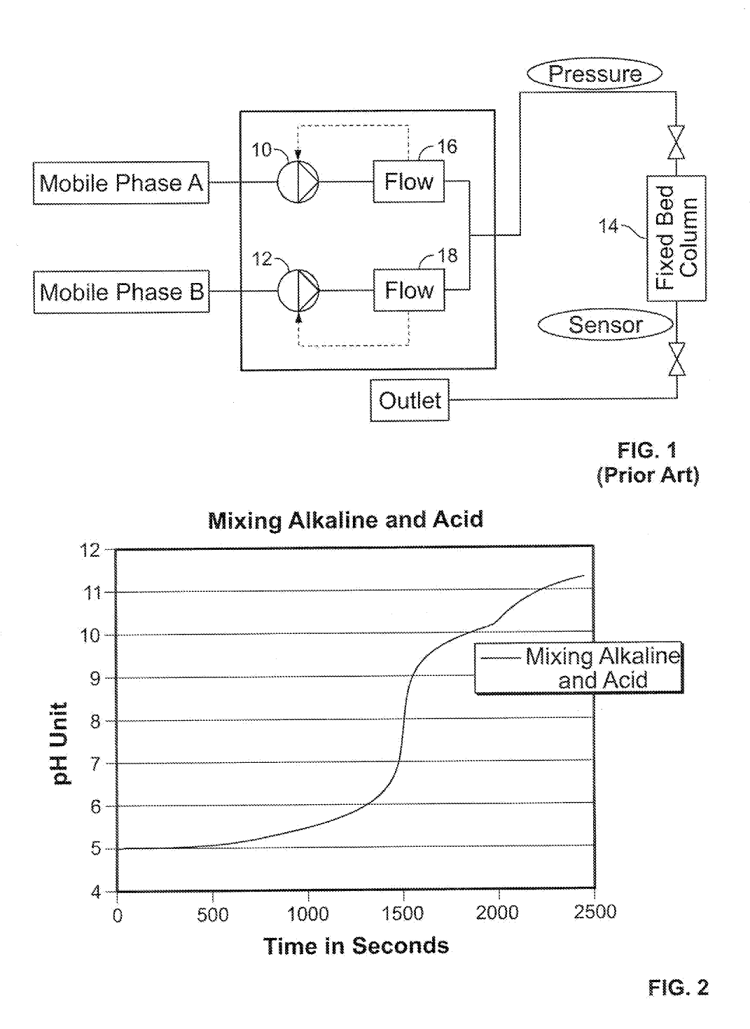

### DETAILED DESCRIPTION OF EMBODIMENTS

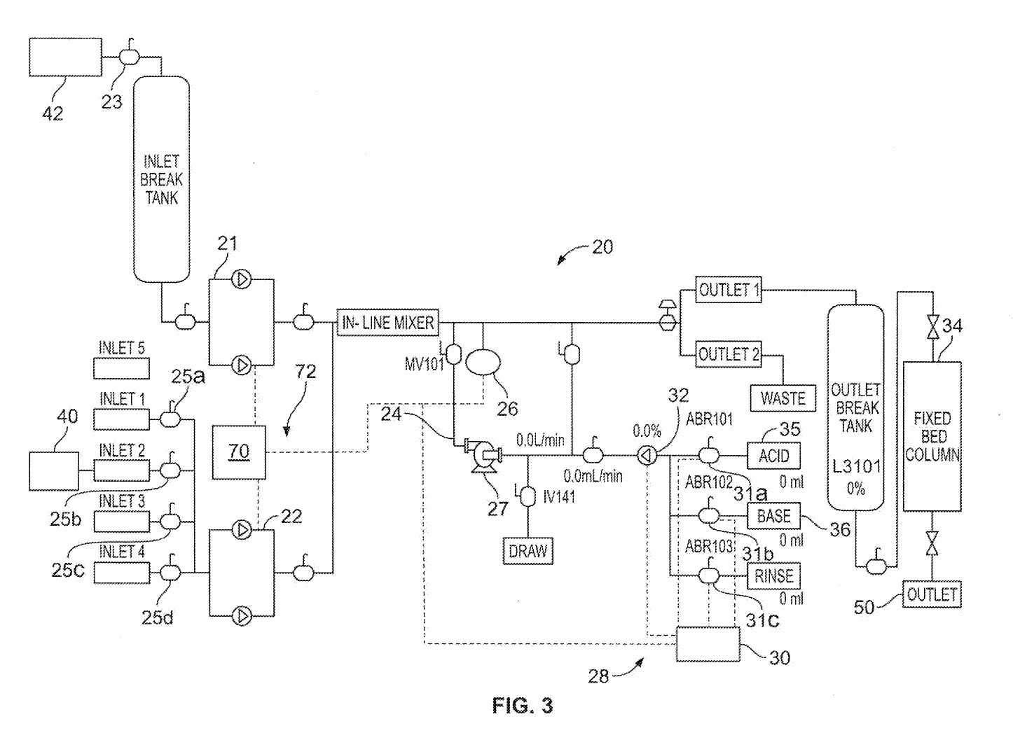

In accordance with the present invention, an accurate blending module or inline buffer dilution device (IBD), such as the one indicated in general 20 in FIG. 3, and in commonly assigned U.S. Pat. No. 7,515,994 to Bellafiore et al. can be used to produce a linear pH gradient in blended liquids for protein separation and purification from a protein solution loaded in a liquid chromatography column. The contents of U.S. Pat. No. 7,515,994, and related U.S. Pat. No. 7,072,742 and U.S. Patent Application Publication No. U.S. 2008/0279038, are hereby incorporated by reference.

FIG. 3 shows a piping and instrumentation diagram of the IBD device 20 in an embodiment of the system of the invention. Either an acid or base/alkali is connected to primary pump 21, and the other to concentrate pump 22. A primary inlet valve 23 communicates with the inlet of the primary pump 21, which may be a water for injection (WFI) pump. A number of concentrate inlet valves 25a-25d communicate with the inlet of the concentrate pump 22. The acidic and alkaline solutions are pumped into a mixing loop 24, which preferably features recirculation pump 27, where the resulting titration solution can be instantaneously analyzed by a controller 30 via a pH sensor 26, so that an inline process pH analyzer is formed. The sensor 26 interfaces with the controller 30 which interfaces with automated titration valves 31a-31c and a titration pump 32 so that a process control feedback loop, indicated in general at 28 in FIG. 3, is formed.

Controller 30 may be, as an example only, a programmable logic controller (PLC). In accordance with the user-defined pH parameters, the controller 30, via the pH sensor 26, continually monitors and adjusts the acidity of the solution in mixing loop 24 by manipulating the automated titration valves 31a-31c and controlling the titration pump 32 so as to pump respective acid or base to the mixing loop. Consequently, a generally linear pH gradient can be created to satisfy the user's requirements by operation of the titration pump 32 and configuration of valves 31a-31c. In accordance with an embodiment of the method of the present invention, the user requirements are to provide a linear pH gradient during delivery of the blended liquid to liquid chromatography column 34 for protein separation and purification from a protein solution loaded in the column.

Controller 30 is preferably configured as a proportional-integral-derivative (PID) controller to control the titration valves 31a-31c and the titration pump 32 and to incorporate the user-defined set points into the IBD device 20 via the controller software. As is known in the art, by tuning the three parameters in the PID controller algorithm, the controller can provide control action designed for specific process requirements. The response of the controller can be described in terms of the responsiveness of the controller to an error, the degree to which the controller overshoots the set point and the degree of system oscillation.

In accordance with an embodiment of the system and method of the present invention, the IBD device 20 of FIG. 3 is connected to a chromatography column 34 packed with a cation exchange resin (available, for example, from Tosoh Biosciences). As an example only, the chromatography column may have a 10 cm inner diameter. It should be understood that an anion exchange resin may be used as an alternative to the cation resin (i.e. any ion exchange resin may be used in the invention). As explained in greater detail below, in an embodiment of the invention, acetic acid (available, for example, from Amresco) and ammonium hydroxide (available, for example, from JT Baker) are used as the acid and base/alkali, 40 and 42 in FIG. 3, respectively, to separate and purify two proteins, for example, Ribonuclease A and Trypsinogen (available, for example, from Worthington Enzymes and Sigma Aldrich, respectively).

Using the IBD device 20 of FIG. 3, a supply of an acetic acid (20 mM) solution 40 is connected to the concentrate pump 22 via inlet valve 25b, which is opened, and the concentrate pump 22 is run with the same starting and ending pump frequency of 12.3%. A supply of ammonium hydroxide (10 mM) 42 is connected to the primary pump 21, via inlet valve 23, which is opened, and the primary pump 21 is operated with a starting pump frequency of 14% and an ending frequency of 10%. Aciditiy of the solution is continually adjusted, via pH sensor 26 and controller 30, with ammonium hydroxide (100 mM), a supply of which is indicated at 36 in FIG. 3, delivered from the titration pump 32 with a starting pump frequency of 10% and ending with 100%. Titration valve 31b is open, but valves 31a and 31c are closed. In the first five minutes, the pH of the blend is maintained at 6 followed by a linear gradient run to pH 10.3 over 30 minutes.

It should be noted that the starting and ending pump frequencies are dependent on pump strength, which are 300 liters/hour for primary pump 21 and 150 liters/hour for concentrate pump 22 in the embodiment illustrated. For example, if the strength of primary pump 21 were changed from 300 liters/hour to 400 liters/hour, a new pump frequency would be required to obtain a linear pH gradient. In addition, if the molarity of the solution were changed, for example, from 20 mM to 40 mM, the pump frequency would also have to be adjusted.

To provide a comparison to the method of the above paragraph, a pH gradent was generated using the IBD device of FIG. 3, but without using the feedback loop 28 and titration pump 32. More specifically, the orientation of the pumps 21 and 22 and the concentration of the mobile phases was the same as the linear pH gradient of the above paragraph, except that the feedback control for pH adjustment was idle. While the starting frequency of the concentrate pump 22 was 29.3% and 7.39% at the end, the primary pump 21 was raised from the initial 10% to final 29.45% over 40 minutes. The initial pH was just below 5, and the final pH was about 11.

Of the two proteins, Trypsinogen, has an isoelectric point (pI) equal to 8.3, and Ribonuclease A, has a pI equal to 9.4. For both of the above runs, 50 mg of each protein were dissolved in 20 mM acetic acid, pH 4, and 100 mM sodium chloride. The mixture was loaded onto the column 34 packed with cation exchange resins. Before loading, the column was pre-equilibrated with a pH 6 starting buffer.

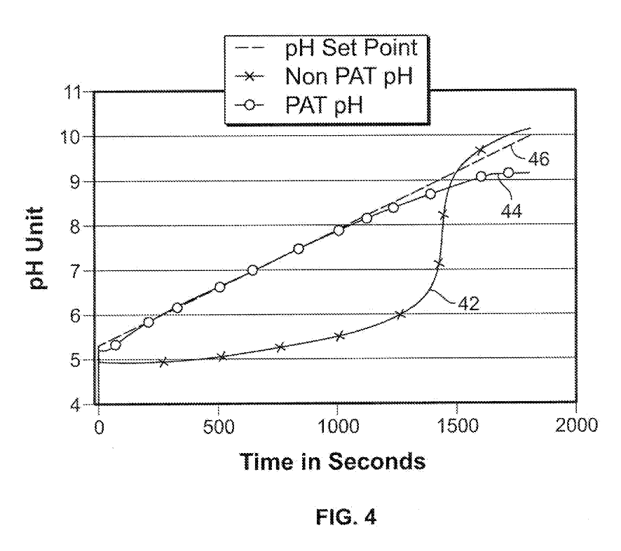

As stated previously, in each run, the 20 mM acetic acid, a weak acid, was titrated with 10 mM ammonium hydroxide, a weak base, to create a pH curve. FIG. 4 shows the results of the two runs. Simply blending acid and alkali without pH adjustment and control via the feedback loop 28 and titration pump 32 (i.e. a passive IBD device) a typical sigmoid titration curve was generated as shown by the X-line 42 in FIG. 4. The steep rise of the pH value from 6.5 to 9.5 makes it difficult to resolve proteins whose pI would be within this range. In contrast, active use of the IBD device 20, that is, using the feedback loop 28 and titration pump 32, was able to develop, and deliver to column 34, a linear pH gradient, illustrated by O-line 44 in FIG. 4. The interfaced process control feedback loop 28 in the active IBD device 20 method constantly analyzed and adjusted the pH of the solution in the loop 24, and referenced the current process value to the user-defined pH target set point in the controller 30 at any given time (line 46 in FIG. 4). More specifically, the controller 30, based on readings from the pH sensor 26, sent a signal to adjust the speed of the titration pump 32 so that the dynamic pH value in the mixing loop 24 was continually adjusted towards the pre-determined set-point.

As noted above, two proteins, Trypsinogen and Ribonuclease A, with similar pI, were chosen to illustrate the effectiveness of the pH control in the active IBD device system and method, which is best shown in FIG. 5, where the elusion behaviors of the two proteins are illustrated. In the case of the passive IBD device 20 operation, the sharp rise of pH from 6.5 to 9.5 within a few minutes caused both proteins to be de-sorbed and to elute from the column almost together through outlet 50 (FIG. 3). As a result, resolution was not good as evidenced by the line 52 in FIG. 5. On the other hand, the gentle pH increase through the active IBD device pH control selectively and slowly changed the ionic characters of the two proteins. As a result, both proteins were baseline separated through outlet 50 as shown by line 54 in FIG. 4.

The embodiment of the system and method of the present invention illustrated in the figures and described above, that is, where an active IBD device was used, was able to produce a generally linear pH gradient. By having an inline process pH analyzer (pH sensor 26 and controller 30) included within process control feedback loop 28, two proteins, Trypsinogen and Ribonuclease A, were successfully resolved. The technology of the present invention could be extended to separate other proteins having pI similar to one another or to remove high molecular weight aggregates from the parent monomer.

While the embodiment of the system and method of the present invention illustrated in FIGS. 3-5 and described above blends/mixes three liquids or solvents, the present invention may be constructed and practiced in an alternative embodiment where a linear pH gradient is obtained by mixing two liquids or solvents, provided, for example, via primary pump 21 and concentrate pump 32 of FIG. 3. The system of FIG. 3 may be used in such an embodiment with the titration pump 32 being inactive/idle.

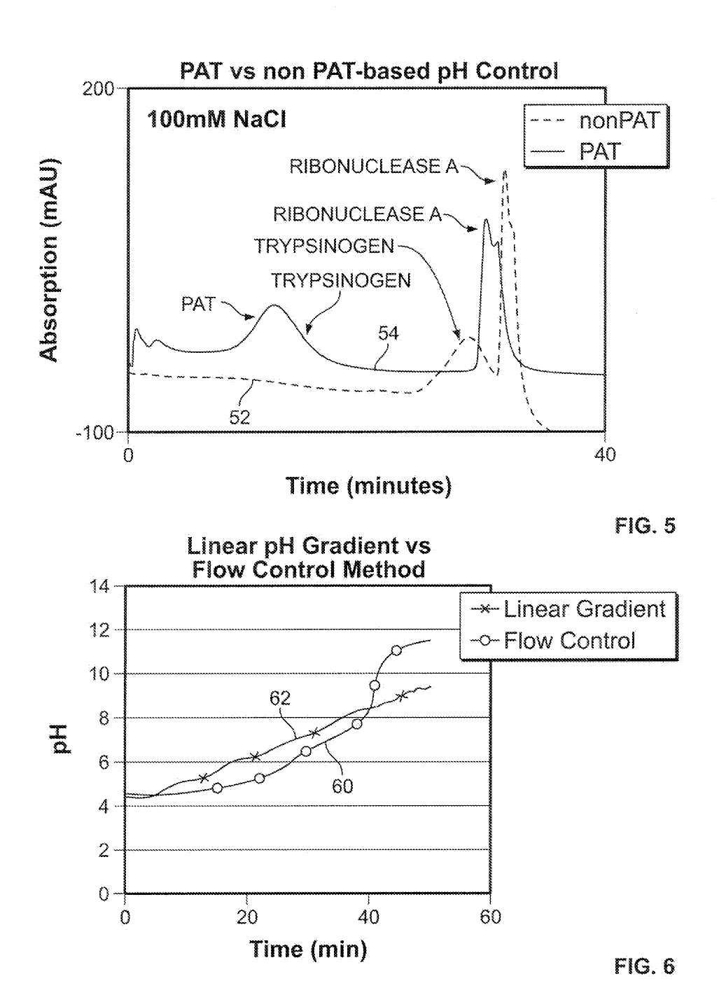

An example of the results of such a two-liquid approach is provided in FIG. 6. In the two-solvent system used to generate FIG. 6, acetic acid (40 mM), pH 4, is provided via primary pump 21, and trisodium phosphate (20 mM), pH 12, is provided via concentrate pump 22. To achieve the “Linear Gradient”, illustrated in the graph of FIG. 6 at 62, the solvents from pumps 21 and 22 are pumped into the mixing loop 24, where the resulting solution is instantaneously analyzed by a controller 70 connected to pH sensor 26 to form an inline process pH analyzer. As illustrated in FIG. 3, controller 70, which also is preferably configured as a PID controller, interfaces with and controls pumps 21 and 22 so that a process control feedback loop, indicated in general at 72 in FIG. 3, is formed. In accordance with the user-defined pH parameters, the controller 70, via the pH sensor 26, continually monitors and adjusts the acidity of the solution by controlling pumps 21 and 22 to provide acid or base. Consequently, a generally linear pH gradient is created to satisfy the user's requirements by operation of the pumps 21 and 22.

To provide a comparison to the method of the above paragraph, in a “Flow Control” mode of operation, a pH gradent was generated using the IBD device 20 of FIG. 3, but without using the pH sensor 26 or controller 70. More specifically, the orientation of the pumps 21 and 22 and the concentration of the mobile phases was the same as the linear pH gradient run of the above paragraph, except that the feedback control for pH adjustment was idle. As a result, the “Flow Control” curve of FIG. 6, illustrated at 60, was generated. The steep slope starting from pH 8 and rising to pH 11 over 2 minutes for curve 60 should be noted in FIG. 6.

In generating the Linear Gradient result (62) of FIG. 6, primary pump 21 was operated with a starting frequency of 24% and an ending frequency of 10%, while concentrate pump 22 was operated with a start frequency of 10% and an ending frequency of 67% (with a combined flow rate of the two pumps equal to 0.4 L/min.). To generate the Flow Control result (60) of FIG. 6, primary pump 21 was operated with a starting frequency of 64% and an ending frequency of 10%, while concentrate pump 22 was operated with a start frequency of 5% and an ending frequency of 67% (with a combined flow rate of the two pumps equal to 0.4 L/min.).

While the preferred embodiments of the invention have been shown and described, it will be apparent to those skilled in the art that changes and modifications may be made therein without departing from the spirit of the invention, the scope of which is defined by the appended claims.

What is claimed is:

1. A system for protein separation and purification comprising:
a. a mixing device;
b. a primary pump having an inlet in communication with a supply of an acid, an alkali or water, and an outlet in communication with the mixing device;
c. a concentrate pump having an inlet in communication with a supply of an acid or an alkali and an outlet in communication with the mixing device;
d. a liquid chromatography column in communication with the mixing device and packed with an ion exchange resin and loaded with proteins;
e. a process control feedback loop in communication with the mixing device including an inline process pH analyzer so that a pH of a titration solution in the mixing device may be detected and adjusted based on the detected pH;
whereby the titration solution is provided in the mixing device by the primary and concentrate pumps and the pH of the titration solution in the mixing device is adjusted by the feedback loop based on the pH detected by the inline process pH analyzer to provide a generally linear pH gradient to the chromatography column so that the proteins are separately eluded from the liquid chromatography column.

2. The system of claim 1 further comprising a recirculation pump in the mixing device.

3. The system of claim 1 wherein the process control feedback loop includes a titration pump having an inlet in communication with a supply of acid or alkali and an outlet in communication with the mixing device so that the pH of the titration solution in the mixing device is adjusted by acid or alkali from the titration pump.

4. The system of claim 3 wherein the process control feedback loop includes a plurality of automated titration valves in communication with the inlet of the titration pump.

5. The system of claim 4 wherein the inline process pH analyzer includes a pH sensor in communication with the mixing device and a controller in communication with the pH sensor, the titration pump and the automated titration valves.

6. The system of claim 3 wherein the inline process pH analyzer includes a pH sensor in communication with the mixing device and a controller in communication with the pH sensor and the titration pump.

7. The system of claim 6 wherein the controller is a programmable logic controller.

8. The system of claim 6 wherein the controller is configured as a PID controller.

9. The system of claim 1 wherein the inline process pH analyzer includes a pH sensor in communication with the mixing device and a controller in communication with the pH sensor.

10. The system of claim 9 wherein the controller is a programmable logic controller.

11. The system of claim 9 wherein the controller is configured as a PID controller.

12. The system of claim 1 wherein the primary pump is a WFI pump.

13. The system of claim 1 wherein the process control feedback loop is in communication with at least one of the primary pump or the concentrate pump so that the pH of the titration solution in the mixing device is adjusted by acid or alkali from the primary pump and/or acid or alkali from the concentrate pump.

14. The system of claim 13 wherein the inline process pH analyzer includes a pH sensor in communication with the mixing device and a controller in communication with the pH sensor, and at least one of the primary pump or the concentrate pump.

15. The system of claim 14 wherein the controller is a programmable logic controller.

16. The system of claim 14 wherein the controller is configured as a PID controller.

17. The system of claim 1 wherein the mixing device includes a mixing loop.

18. The system of claim 1 wherein the proteins include at least two proteins having a first isoelectric point and a second protein having a second isoelectric point.

19. A method for separating and purifying proteins comprising the steps of:
a. providing a mixing device, a primary pump in communication with the mixing device, a concentrate pump in communication with the mixing device, a liquid chromatography column in communication with the mixing device and a process control feedback loop in communication with the mixing device including an inline process pH analyzer so that a pH of a titration solution in the mixing device may be detected and adjusted based on the detected pH;
b. packing the liquid chromatography column with an ion exchange resin;
c. loading the chromatography column with a protein solution containing proteins;
d. supplying an acid or an alkali to the mixing device using the primary pump;
e. supplying an acid or an alkali to the mixing device using the concentrate pump so that the titration solution is created in the mixing device; and
f. adjusting the titration solution in the mixing device using the feedback loop based on the pH detected by the inline process pH analyzer to provide a generally linear pH gradient to the chromatography column so that the proteins are separately eluded from the liquid chromatography column.

20. The method of claim 19 wherein the process control feedback loop includes a titration pump and wherein the titration solution in step f. is adjusted by acid or alkali provided by the titration pump.

21. The method of claim 19 wherein the process control feedback loop is in communication with at least one of the primary pump or the concentrate pump and wherein the titration solution in step f. is adjusted by acid or alkali provided by the primary pump and/or the concentrate pump.

22. The method of claim 19 wherein a frequency of one of the primary pump or the concentrate pump is held steady during steps d. and e. while a frequency of the other one of the primary pump or the concentrate pump is varied during steps d. and e.

23. The method of claim 19 wherein the mixing device includes a mixing loop.

24. The method of claim 19 wherein the proteins include at least two proteins having a first isoelectric point and a second protein having a second isoelectric point.
